(19) United States
(12) Patent Application Publication
(10) Pub. No.: US 2017/0196924 A1
(43) Pub. Date: Jul. 13, 2017

(54) COMPOSITION FOR SUPPORTING CELLULAR STRUCTURE AND ADHESION

(71) Applicant: All Natural Protectors, LLC, St. Louis, MO (US)

(72) Inventor: Timothy J. O'Brien, Little Rock, AR (US)

(21) Appl. No.: 15/380,573

(22) Filed: Dec. 15, 2016

Related U.S. Application Data
(60) Provisional application No. 62/276,856, filed on Jan. 9, 2016.

(51) Int. Cl. A61K 36/48; A23J 3/14; A23L 33/105; A61K 36/28; A61K 47/26; A61K 31/12; A61K 9/00; A61K 38/16; A61K 47/46; A23J 3/16; A61K 36/42

(52) U.S. Cl. CPC A61K 36/48; A23J 3/16; A23J 3/14; A23L 33/105; A61K 36/28; A61K 36/42; A61K 31/12; A61K 9/0056; A61K 9/0053; A61K 38/168; A61K 47/46; A61K 47/26; A23V 2002/00

(57) ABSTRACT

The present invention is a composition comprising: (a) protein selected from the group consisting of soy protein and pea protein; (b) sunflower seed protein; (c) turmeric; and (d) bitter melon. The composition is useful as a dietary supplement, a food, a complementary medicine or a therapeutic agent. Furthermore, the composition may be prepared in any form including, for example, powder, emulsion, liquid, capsule, pill and food items such as nutrition bars.

### BACKGROUND

The soybean or soya bean is a species of legume native to East Asia, widely grown for its edible bean which has numerous uses. People have been eating soybeans for almost 5,000 years. Unlike most plant foods, soybeans are high in protein. The plant is classed as an oilseed rather than a pulse by the UN Food and Agricultural Organization (FAO). Soybeans were first introduced to North America in 1765, by Samuel Bowen, a former East India Company sailor and for the next 155 years the crop was grown primarily for forage. William Morse is considered the father of modern soybean agriculture in America. He and Dr. C. V. Piper took what was an unknown Asian peasant crop in 1910 and transformed it into a ‘golden bean’ for America. The soybean has become one of America's largest and most nutritious farm crops. Prior to the 1920s in the USA, the soybean was mainly a forage crop, a source of oil, meal (for feed) and industrial products, with very little used as food, but it took on an important role after World War I.

Today, researchers are interested in both the nutritional value and the potential health benefits of soy. Several clinical studies of people who eat a lot of soy foods, such as people in Asia, suggest that soy may help reduce menopausal symptoms, and lower the risk of heart disease and osteoporosis. Researchers are also examining whether eating soy foods can help prevent hormone-related cancers, such as breast, prostate, and uterine cancer. Isoflavones are phytoestrogens—chemicals that act like the hormone estrogen. Soy phytoestrogens are very complex, and they can either strengthen or reduce the effects of estrogen in the body. Soy contains other nutrients as well. Some researchers believe that the healthful benefits of soy foods may come from the combination of these nutrients and not just the isoflavones.

Soy protein isolate is a dry powder food ingredient that has been separated or isolated from the other components of the soybean, making it 90 to 95 percent protein and nearly carbohydrate and fat-free.

The sunflower seed is the fruit of the sunflower. The sunflower is a tall, erect, herbaceous annual plant belonging in the family of Asteraceae, in the genus, Helianthus. Its botanical name is Helianthus annuus. It is native to Middle American region from where it spread as an important commercial crop all over the world by the European explorers. At present, Russian Union, China, USA, and Argentina are the leading producers of sunflower crop. Sunflower seeds are an excellent source of vitamin E, the body's primary fat-soluble antioxidant. Phytosterols are compounds found in plants that have a chemical structure very similar to cholesterol, and when present in the diet in sufficient amounts, are believed to reduce blood levels of cholesterol, enhance the immune response and decrease risk of certain cancers. Sunflower seeds are high in unsaturated fat, which ran help lower blood pressure and total cholesterol.

Turmeric (Curcuma longa) is a rhizomatous herbaceous perennial plant of the ginger family, Zingiberaceae. It is native to southeast India, and needs temperatures between 20° C. and 30° C. (68° F. and 86° F.) and a considerable amount of annual rainfall to thrive. Plants are gathered annually for their rhizomes, and propagated from some of those rhizomes in the following season. The leading commercial producers of turmeric include India, Indonesia, China, the Philippines, Taiwan, Haiti and Jamaica.

In India, turmeric has been used traditionally for thousands of years as a remedy for stomach and liver ailments, as well as topically to heal sores due to its supposed antimicrobial property. In the Siddha system (since c. 1900 BCE) turmeric was a medicine for a range of diseases and conditions, including those of the skin, pulmonary, and gastrointestinal systems, aches, pains, wounds, sprains, and liver disorders. A fresh juice is commonly used in many skin conditions, including eczema, chicken pox, shingles, allergy, and scabies. One of the most comprehensive summaries of turmeric studies to date was published by the respected ethnobotanist James A. Duke, PhD, in the October, 2007 issue of Alternative & Complementary Therapies, and summarized in the July, 2008, issue of the American Botanical Council HerbClip. Reviewing some 700 studies, Duke concluded that turmeric appears to outperform many pharmaceuticals in its effects against several chronic, debilitating diseases and does so with virtually no adverse side effects.

One of the active compounds of turmeric is curcumin, which is believed to have a wide range of biological effects including anti-inflammatory, antioxidant, antitumour, antibacterial, and antiviral activities. It is a major ingredient in Indian curries, and makes American mustard yellow. Evidence is accumulating that this brightly colored relative of ginger is a promising disease-preventive agent as well, probably due largely to its anti-inflammatory action. In numerous studies, curcumin's anti-inflammatory effects have been shown to be comparable to the potent drugs hydrocortisone and phenylbutazone as well as over-the-counter anti-inflammatory agents such as ibuprofen.

Epidemiological studies have linked the frequent use of turmeric to lower rates of breast, prostate, lung and colon cancer; laboratory experiments have shown curcumin can prevent tumors from forming; and research conducted at the University of Texas suggests that even when breast cancer is already present, curcumin can help slow the spread of breast cancer cells to the lungs in mice. Much of its recent popularity is owed to the recent research that has highlighted its therapeutic properties.

Momordica charantia, known as bitter melon, bitter gourd, bitter squash or balsam-pear in English, has many other local names. It is a tropical and subtropical vine of the family Cucurbitaceae, widely grown in Asia, Africa, and the Caribbean for its edible fruit, which is extremely bitter. Its many varieties differ substantially in the shape, size and bitterness of the fruit. It is a member of the same family as squash, watermelon, cantaloupes, and cucumber. Bitter melon has been used in various Asian and African herbal medicine systems for a long time. In Turkey, it has been used as a folk remedy for a variety of ailments, particularly stomach complaints. In traditional medicine of India different parts of the plant are used to relieve diabetes, as a stomachic, laxative, antibilious, emetic, anthelmintic agent, for the treatment of cough, respiratory diseases, skin diseases, wounds, ulcer, gout, and rheumatism.

The vegetable is very low in calories and notably contains polypeptide-P, which is considered a plant insulin because it is known to lower blood sugar levels in animal models. In addition, it contains a hypoglycemic agent called charantin. Charantin increases glucose uptake and glycogen synthesis inside the cells of liver, muscle and adipose tissue. Together, these compounds have been thought to be responsible for reduction of blood sugar levels in the treatment of type-2 diabetes. In vitro and animal studies indicate anti-cancer, anti-viral, and lipid lowering effects. Bitter melon juice has been shown to slow pancreatic tumor xenografts in mice (Bitter melon juice activates cellular energy sensor AMP-activated protein kinase causing apoptotic death of human pancreatic carcinoma cells. Kaur M, Deep G, Jain A K, Raina K, Agarwal C, Wempe M F, Agarwal R. Carcinogenesis. 2013 July; 34(7):1585-92).

Phytonutrients (phytochemicals) are chemicals that occur naturally in plants. Some are responsible for color and other organoleptic properties, such as the deep purple of blueberries and the smell of garlic. The term is generally used to refer to those chemicals that may have biological significance or activity, for example carotenoids or flavonoids, but are not established as essential nutrients. Exemplary phytonutrients (phytochemicals) found in soy, pea, sunflower seed and turmeric include, but are not limited to, the following:

| Phytochemicals | Source | Phytochemicals | Source |
|---|---|---|---|
| Bowman-Birk | Soy/Pea | Sunflower Trypsin | Sunflower |
| Inhibitor (BBI) (SF) | | Inhibitor-1 | |
| Soy/Pea (SFTI-1) | | Kuntz Inhibitor | |
| Isoflavones: | Soy | Phytosterols | Sunflower |
| Genistein | | Curcumin | Turmeric (T) |
| Daidzein | | Demethoxycurcumin | Turmeric |
| Glycitein | | Bismethoxycurcumin | Turmeric |
| Phytic Acid | Soy/SF | COX-2 Inhibitor | Turmeric |
| Beta-Sitosterol | Soy | Glycosides: | Bitter |
| Lunasin | Soy | Momordin | |
| Melon | | Charantin | |
| Phenolic Acids | Pea/SF | Alkaloid: Momordicin | Bitter |
| Lignans | Soy/Pea/SF | Polypeptide-P | Bitter |
| Flavanoids | Pea | Curcurbitane | Bitter |
| Melon | | triterpenoids | |
| Triterpenoids | Pea/SF | MCTI-I/MCTI-II | Bitter |
| Melon | | Protein MAP 30 | Bitter |
| Polyphenols | Pea/T | | |
| Melon | | | |
| Saponins | Soy/Pea/SF | | |
| Melon | | | |
| Galactose | Pea | | |
| oligosaccharides | | | |
| Melon | | | |

Health benefits associated with these phytonutrients include, but are not limited to, the following:
Anti-oxidant properties
Anti-inflammatory properties
Anti-cancer properties
May lower blood pressure
May improve blood sugar control in diabetes
May improve the ratio of LDL (“bad”) verses HDL (“good”) cholesterol levels
May offer cardiovascular protection properties
May improve detoxification functions.

Studies of the many phytonutrients associated with plants is a growing field in the natural medicine approach to quality of life.

### SUMMARY

One embodiment of the present invention is a composition for improvement of health, supporting cellular tissue structure and supporting cellular adhesion. It comprises ingredients that are beneficial for the body's health, including, in some examples, soy protein or pea protein, sunflower seed, turmeric, and bitter melon. These have been combined to make a food, a dietary supplement, a complementary medicine or a therapeutic agent.

One aspect of these listed components is that they all include protease inhibitors that are believed to inhibit growth and metastasis of tumors in humans.

Endogenously produced proteases of tumor cells are important for tumor growth and especially for metastasis. Metastasis is almost always required for a cancer to kill a person. So inhibition of metastasis can extend patients lives, and this may be achieved by inhibitors of proteases. Thus, the composition is believed to prevent or delay tumor growth and metastasis in patients, and possibly prevent cancer in humans who have not been diagnosed with cancer.

The composition promotes cellular adhesion in the sense that it inhibits proteases and proteases are involved in cancer cells metastasizing, that is, escaping from the primary tumor location by de-adhering from that location. It promotes cellular tissue structure in the sense that proteases break down tissue structure, which in cancer allows metastases to invade new tissue sites.

One embodiment of the invention is a composition comprising: (a) protein selected from the group consisting of soy protein and pea protein; (b) sunflower seed protein; (c) turmeric; and (d) bitter melon.

Another embodiment provides a method of inhibiting cancer metastasis or tumor growth comprising ingesting or orally administering the composition.

Another embodiment provides a method of preventing the occurrence of cancer comprising ingesting or orally administering the composition.

Another embodiment provides a method of promoting breast, prostate, colon, ovary, pancreas, or lung health comprising ingesting or orally administering the composition.

### DETAILED DESCRIPTION

The present invention is a composition comprising either soy protein or pea protein, and sunflower seed protein, turmeric and bitter melon. In certain embodiments of the invention, the soy protein is whole soybean, ground soybean, or protein substantially isolated from soybean. In other embodiments, soy protein may be replaced or supplemented by pea protein which may be either whole pea, ground pea, or protein substantially isolated from pea. Additionally, sunflower seed may be whole sunflower seed, ground sunflower seed or protein substantially isolated from sunflower seed. Turmeric may be added to the composition in its raw form (as a powder formed from dry root, or particles or pieces of dried turmeric root) or as substantially isolated protein from turmeric root or as purified curcumin. Bitter melon may be added to the composition in its raw form, a dried powder, or as protein substantially isolated from bitter melon. The bitter melon component may be derived from bitter melon fruit or whole bitter melon plant.

Each of the components of the composition contains protease inhibitors. Soy protein includes Bowman-Birk inhibitor (BBI) and Kuntz inhibitor. Sunflower contains Sunflower Trypsin Inhibitor-1 (SFTI-1). Turmeric contains curcumin, which has been shown to be a metaloprotease inhibitor (Thaloor, D. et al., 1998, Cell Growth & Differentiation 9:305-312). Bitter melon also contains protease inhibitors (Telang M et al. Phytochemistry. 2003 July; 63(6):643-52). Endogenously produced proteases of tumor cells are important for tumor growth and especially for metastasis. Among the proteases expressed by cancer cells are CA125, hepsin, matriptase, and the kallikreins. Metastasis is almost always essential for a cancer to kill a person. So inhibition of metastasis can extend patients lives, and this may be achieved by inhibitors of proteases.

A variety of proteases are produced by different cancers, and even by the same cancer in a single person. So ingesting a variety of protease inhibitors is believed by the inventors to be important to have the best chance to inhibit the protease or proteases that may be produced by the tumor in a particular human cancer patient, or by an undetected microtumors in a person not yet diagnosed with cancer.

Protease inhibitors in particular, as well as other health-promoting ingredients in the components of the composition, can be inactivated or damaged by heat. Thus, it is preferable but not essential, that the components of the composition are not prepared by a process that includes high heat. Thus, in specific embodiments of the invention, the soy, pea, sunflower seed, or bitter melon components of the composition are cold processed, that is, they are not prepared or isolated by a process that involves heating above 70 degrees C. during processing to prepare the component.

In one embodiment of the present invention, (a) soy protein powder or pea protein powder or a combination thereof, (b) ground sunflower seeds, (c) turmeric root powder or curcumin, and (d) bitter melon powder are combined to make a powder product. Such powder product can be considered a food, a dietary supplement, a complementary medicine or a therapeutic agent. The powder composition may be consumed directly. Therefore, one embodiment of the invention is a powder. In some variation of the invention, the powder form of the composition may be mixed or blended with a liquid, such as, for example, water, milk, or a juice such that another embodiment of the invention is a drink.

Other embodiments of the composition include bars and biscuits.

A suitable soy protein is available as Bob's Red Mill isolated soy protein powder (SKU: 1512C28) from Bob's Red Mill Natural Foods, Milwaukie, Oreg.

A suitable pea protein is available as pea protein powder (UPC: 849720014214) from BulkSupplements.com, Henderson, Nev.

A suitable sunflower seed is available as Bob's Red Mill simply raw sunflower seeds (SKU: 1526C20) from Bob's Red Mill Natural Foods, Milwaukie, Oreg.

A suitable turmeric is available as turmeric root powder organic (SKU: 209975-51) from Starwest Botanicals, Inc. Sacramento, Calif. A suitable curcumin as the turmeric is available as BulkSupplements Pure Curcumin 95% Natural Turmeric Extract Powder (ASIN: B00GHY362C; UPC: 849720002426) from BulkSupplements.com in Henderson, Nev.

A suitable bitter melon is available as bitter melon powder (SKU: 201069-51) from Starwest Botanicals, Inc. Sacramento, Calif.

In some embodiments of the invention, the composition may comprise a sweetener. Exemplary sweeteners are inulin, stevia, saccharine, sucrose, dextrose, fructose, honey, and any other known sweetener.

In various embodiments of the invention, the composition may further comprise a flavoring agent. Examples of such flavoring agents are chocolate, vanilla, strawberry and butterscotch flavoring.

In specific embodiments of the compositions, the composition comprises 25% to 80%, on a dry weight basis, of soy or pea or a combination of soy and pea. The soy and pea comprise soy protein and pea protein, respectively, but they may comprise other components, especially fat and carbohydrate. They may be whole ground soy or pea, or purified soy protein powder or pea protein powder. In more specific embodiments, the composition comprises 30% to 75%, on a dry weight basis, soy or pea or a combination of soy and pea.

In specific embodiments, the composition may comprise on a dry weight basis 5% to 50% sunflower seed (meaning the whole seed or components derived from sunflower seed, whether the components are protein, fat, or carbohydrate). In more specific embodiment, the composition comprises on a dry weight basis 10% to 50% sunflower seed.

In specific embodiments, the composition may comprise on a dry weight basis 0.1% to 30% turmeric (meaning any component derived from turmeric plant). If the turmeric is powder or dried particles of whole turmeric plants or root, the composition preferably comprises 2% to 30% turmeric. If the turmeric is curcumin, the composition preferably comprises 0.1% to 3% curcumin.

In specific embodiments, the composition may comprise on a dry weight basis 2% to 30% bitter melon (meaning any component derived from bitter melon plant).

### EXAMPLE

One embodiment of the composition contains:

15 grams Soy Protein OR Pea Protein (OR a combination of the two),

8 grams of ground sunflower seeds,

3 grams of turmeric root powder, and

5 grams of bitter melon powder.

The composition may further comprise one or more flavors and/or one or more sweeteners. For instance, it may comprise cocoa and sugar, or vanilla flavor and stevia.

What is claimed is:

1. A composition comprising: (a) protein selected from the group consisting of soy protein and pea protein; (b) sunflower seed protein; (c) turmeric; and (d) bitter melon.

2. The composition of claim 1 wherein each of components (a), (b), (c), and (d) is a dried powder.

3. The composition of claim 1 wherein each of components (a), (c), and (d) is a dried powder, and component (b) is whole sunflower seeds.

4. The composition of claim 1 wherein component (d) is a dried powder from whole bitter melon fruit or whole bitter melon plant.

5. The composition of claim 1 wherein component (c) is curcumin.

6. The composition of claim 1 wherein the composition is at least 30% by dry weight soy or pea, at least 1% sunflower seed protein by dry weight, at least 1% turmeric by dry weight, and at least 2% bitter melon by dry weight.

7. The composition of claim 5 wherein the composition is at least 30% by dry weight soy or pea, at least 1% sunflower seed protein by dry weight, at least 0.1% turmeric by dry weight, and at least 2% bitter melon by dry weight.

8. The composition of claim 1 further comprising at least one of a sweetener and a flavoring agent.

9. The composition of claim 8 wherein the composition comprises a flavoring agent selected from the group consisting of vanilla flavor, chocolate flavor, strawberry flavor, and butterscotch flavor.

10. The composition of claim 1 wherein said composition is a powder, liquid, emulsion, or bar.

11. The composition of claim 1 wherein the composition is a tablet, a capsule, or a bulk powder.

12. The composition of claim 1 wherein none of components (a), (b), or (d) is heated above 70 degrees C. during processing to prepare the component.

13. A dietary supplement comprising the composition of claim 1.

14. A food comprising the composition of claim 1.

15. A therapeutic agent comprising the composition of claim 1.

16. A complementary medicine comprising the composition of claim 1.

17. A method of inhibiting cancer metastasis or tumor growth comprising ingesting or orally administering the composition of claim 1.

18. A method of preventing the occurrence of cancer comprising ingesting or orally administering the composition of claim 1.

19. A method of promoting breast, prostate, colon, ovary, pancreas, or lung health comprising ingesting or orally administering the composition of claim 1.

20. A method of promoting cellular structure or cell adhesion comprising ingesting or orally administering the composition of claim 1.
